(19) United States
(12) Patent Application Publication
(10) Pub. No.: US 2020/0297722 A1
(43) Pub. Date: Sep. 24, 2020

(54) pH-Modulated Formulations for Pulmonary Delivery

(71) Applicant: GRIFOLS, S.A., Los Angeles, CA (US)

(72) Inventors: David C. Cipolla, San Ramon, CA (US); Igor Gonda, San Francisco, CA (US)

(73) Assignee: GRIFOLS, S.A., Los Angeles, CA (US)

(21) Appl. No.: 16/896,132

(22) Filed: Jun. 8, 2020

Related U.S. Application Data
(63) Continuation of application No. 15/218,971, filed on Jul. 25, 2016, now abandoned.
Application No. 15/218,971 is a continuation of application No. 12/693,739, filed on Jan. 26, 2010, now abandoned.
(60) Provisional application No. 61/153,556, filed on Feb. 18, 2009.

(51) Int. Cl. A61K 31/496; A61K 31/43; A61K 31/545; A61K 31/65; A61K 33/24; A61K 9/00; A61K 9/12

(52) U.S. Cl. CPC A61K 31/496; A61K 31/43; A61K 31/545; A61K 9/12; A61K 33/24; A61K 9/0078; A61K 9/007; A61K 31/65

(57) ABSTRACT

An aerosolizable formulation comprised of a drug, a carrier and pH affecting component is disclosed. The Drug is dissolved in the formulation at a concentration above which it remains in solution at neutral pH. This increases the concentration of the drug in solution making it possible to administer larger amounts of drug with the same or a smaller volume of formulation. When the formulation is aerosolized to small particles and inhaled into human lungs in small volumes (e.g. 0.05 to 0.5 mL) the fluids in the lungs neutralize the formulation causing the drug to participate out of solution. This results in the drug being delivered at a controlled rate below the rate at which drug is administered from a formulation initially at a neutral pH.

### RELATED APPLICATIONS

This application is a continuation of U.S. application Ser. No. 15/218,971, filed Jul. 25, 2016, which is a continuation of U.S. application Ser. No. 12/693,739, filed Jan. 26, 2010, which claims priority to U.S. application Ser. No. 61/153,556, filed Feb. 18, 2009, the disclosure of each of which is hereby incorporated by reference in its entirety.

### FIELD OF THE INVENTION

The invention relates generally to formulations for the aerosolized delivery of drugs and the use of such formulations to obtain characteristics by changing the pH of the formulation in a direction away from neutral and allowing the formulation to become more neutral after administration.

### BACKGROUND OF THE INVENTION

There are a large number of drugs which are generally administered by some type of injection. Although injecting drugs provides a number of advantages, at times, for some patients it is inconvenient and can be painful and may cause transmission of infection. Such drugs may be administered instead via the lung into the systemic circulation to avoid the fear and pain of injections and potential complications with infections. Another reason for administration of drugs by inhalation is if their intended site of action is in the respiratory tract: depositing drugs within the respiratory tract leads to high concentration in the desired organ and relatively low concentrations outside the respiratory tract. This could lead to improved efficacy and safety compared to the administration of drugs for the treatment of respiratory tract by routes other than inhalation.

Gallium Nitrate is a highly water soluble crystalline Gallium source for uses compatible with nitrates and lower (acidic) pH. Nitrate compounds are generally soluble in water. Nitrate materials are also oxidizing agents. When mixed with hydrocarbons, nitrate compounds can form a flammable mixture. Nitrates are excellent precursors for product ion of ultra-high purity compounds and certain catalyst and nanoscale (nanoparticles and nanopowders) materials. All metallic nitrates are inorganic salts of a given metal cation and the nitrate anion. Mechanisms of Therapeutic Activity for Gallium. Lawrence R. Bernstein. Pharmacological Reviews. Vol 50, No 4, pp 665-682 (1998). Available at: http://www.pharmrev.org.

The nitrate anion is a univalent (−1 charge) polyatomic ion composed of a single nitrogen atom ironically bound to three oxygen atoms (Symbol: NO₃) for a total formula weight of 62.05. Gallium Nitrate is generally commercially available in most volumes. High purity, submicron and nanopowder forms are available as is Gallium Nitrate Solution.

A potential problem with formulating drugs for pulmonary delivery is that the formulation must include a relatively high concentration of the drug in order to reduce the volume so that the aerosolized volume can be readily inhaled by the patient in one inhalation or a minimum number of inhalations to obtain a therapeutically effective dose. Another potential problem is that the drug is unstable at neutral pH whereas it is stable at acidic or basic pH. It is important for safety reasons to avoid dramatic changes of the pH at the deposition sites in the lung as this could lead to safety problems. Another potential problem is that upon delivery all of the drug in the formulation is immediately made available to the patient which can mean that too much drug is made available and put into circulation too quickly, i.e. a short Tₘₐₓ and high Cₘₐₓ Further, it may be that the inhaled formulation does not provide any sustained release of drug over time. Formulations of the present invention endeavor to solve some or all of these problems.

### SUMMARY OF THE INVENTION

The invention provides for pulmonary delivery of inhaled compounds in a manner which reduces the administration volume, increases drug stability, and/or provides sustained release of the drug and reduces the rate of absorption into the systemic circulation relative to a conventional formulation for pulmonary delivery which is isotonic and at a neutral pH.

The invention is an aerosolizable liquid solution of a pharmaceutical formulation that is physically and chemically stable. When the formulation comes into contact with the respiratory tract, the formulation undergoes a physico-chemical change with respect to the active drug and/or the excipients, which reduces the solubility of the formulation in the respiratory tract so that the residence in the respiratory tract is increased and the drug concentration in the systemic circulation is reduced. Stated differently, Tₘₐₓ is increased and Cₘₐₓ is decreased.

It is generally believed that inhaled drug formulations must be isotonic and formulated at a neutral pH in order to be compatible with the neutral pH of the Jung fluid and not cause broncho-constriction or cough due to perturbations in the lung fluid pH or tonicity. These side effects have been observed for nebulized therapeutics which deliver relatively large fluid volumes (e.g., 2 to 5 mL) of formulation to the lung. However, if the therapeutic dose can be delivered in a small volume; e.g., in one or a few AERx strip® dosage forms which each typically contain 50 μL, and the formulation buffering capacity is low, then the inhaled dose will not significantly perturb the lung fluid pH or tonicity. Thus, by delivering a small volume of formulation (e.g., 0.05 to 0.5 mL) it is possible to ameliorate or eliminate any side effects due to differences in pH or tonicity.

Compounds which are not very soluble or s table at the neutral pH of the lung but are soluble at higher or lower pH, and are stable at those pHs, are formulated, in accordance with the invention, at a pH where the compound has a higher solubility and/or greater stability. Formulating in this manner makes it possible for a therapeutic dose to be delivered in a reduced solution volume. This assists in making the therapy convenient for chronic administration via the pulmonary route.

One potential benefit of this formulation strategy is that once the droplets deposit in lung fluid they will rapidly equilibrate to the substantially neutral pH of the lung fluid. This causes the drug to exceed its solubility at the neutral pH resulting in the formation of crystals or otherwise causing the drug to precipitate out of solution. This precipitate or crystallized drug provides a depot like release in the lung which increases Tₘₐₓ by 10% or more, or 20% or 100% or more. This increases the efficacy if the site of activity is in the lung, and avoids rapid absorption into the systemic circulation.

A slower absorption rate (increased Tₘₐₓ) reduces side effects related to a high systemic Cₘₐₓ. Of particular interest are drugs which have systemic side effects and/or which exhibit pharmacological activity in the deep lung or alveolar space; e.g., gallium nitrate or its other salts to treat hypercalcemia.

There may be multiple options to enable and optimize delivery of the aforementioned drugs to the deep lung. The options include the choice of aerosol delivery system including nebulizers, solution inhalers, vapor condensation aerosol generators, MDIs or via the use of aerosols containing lower density or geometrically smaller droplets or particles, or via slower inhalation flow rates to reduce impaction in the oropharynx and central airways. Of particular interest is the use of Aradigm's AERx Essence® System and AERx family of devices, as described in U.S. Pat. Nos. 5,497,763; and 6,123,068 and related U.S. and non-U.S. patents and publications all of which are incorporated herein by reference to disclose and describe delivery devices, packets that hold drug and methods of administration.

This invention can be enhanced by the use of specific formulation agents or in combination with other delivery strategies. For example, a variety of formulations, polymers, gels, emulsions, particulates or suspensions, either singly or in combination, could be used to increase the sustained release profile in the deep lung and enhance the delay in systemic absorption. The rate of release can be designed to provide dosing over a period of hours, days or weeks. This can be accomplished in many ways; e.g., by coating the aerosol particles with excipients that dissolve slowly in the aqueous environment of the lung (e.g., PLGA, polymers, etc.) or by coating or encapsulating the drug molecules with excipients that release the drug slowly (e.g., liposomes, surfactants, etc.).

Other formulation strategies also exist for delaying or extending the release profile of the drug in the lung. Even though the same amount of drug may still be delivered to the Jung in these scenarios, the peak drug concentration that is absorbed into the bloodstream after inhalation would be attenuated resulting in a reduction in, or elimination of, the side effect profile. Stated differently, reducing Cₘₐₓ reduces side effects. A potential additional feature of this delivery modality is one of convenience for the patient. The frequency of dosing may also be reduced, thereby potentially increasing patient convenience or compliance to therapy, and thus efficacy. Stated differently, increasing Tₘₐₓ improves convenience and as such patient compliance.

Gallium nitrate can be used to treat high calcium levels as can other compounds known to be used for the treatment of patients with hypercalcemia which may be cancer related hypercalcemia.

There are many patients and indications for which this therapeutic improvement with other drugs may be beneficial, including pulmonary hypertension, lung cancer, cystic fibrosis, bronchiectasis, pneumonia, COPD, asthma, pulmonary fibrosis, and other lung diseases.

There are also many potential drugs which may benefit from this invention including gallium nitrate, pentamidine, treprostinil, iloprost, bronchodilators, corticosteroids, anticholinergics, PDE-4 inhibitors, T cell immunomodulators, antioxidants, selective iNOS inhibitors, P2Y receptor agonists, Interleukin-4, 5, 12, 13, or 18 antagonists, antisense inhibitors, ribozyme therapy, CpG oligonucleotides, protease inhibitors, leukotriene inhibitors and gene therapy.

These and other objects, advantages, and features of the invention will become apparent to those persons skilled in the art upon reading the details of the formulations, methods and devices as more fully described below.

### DEFINITIONS

Cₘₐₓ is the maximum concentration of a drug in the body after dosing.

Tₘₐₓ is the period of time after dosing that it takes for Cₘₐₓ to occur.

Before the present formulations, methods and devices are described, it is to be understood that this invention is not limited to particular formulations, methods and devices described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a drug” includes a plurality of such drugs and reference to “the particle” includes reference to one or more particles and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further , the dates of publication provided may be different from the actual publication dates which may need to be independently confirmed.

### DETAILED DESCRIPTION OF THE INVENTION

A formulation for delivery to a patient's respiratory tract by inhalation is disclosed, wherein the formulation is comprised of a pharmaceutically active drug, a pharmaceutically acceptable carrier and a pH affecting agent which increases solubility of the drug in the carrier and is present in a molarity so as to deviate formulation pH by at least 0.5 log unit and not more than 5.4 log units away from 7.4.

The formulation may be further characterized such that when the formulation is in contact with the patient's respiratory tract fluids for a period of time and under conditions present in a human lung that the formulation moves closer to a pH of 7.4 by 0.5 log unit or more relative to the pH of the formulation prior to administration.

The formulation may be still further characterized such that while in the human lung the drug becomes less soluble as compared to its solubility in the formulation prior to administration.

The formulation may be produced wherein the drug is a gallium salt and wherein the gallium salt is gallium nitrate and wherein the pH effecting agent deviates formulation pH by 0.75 to 4.15 log units away from 7.4 or wherein the pH effecting agent deviates formulation pH by 1.0 to 2.0 log units or more away from 7.4.

The formulation may be produced wherein the drug is an antibiotic such as an antibiotic is selected from the group consisting of a penicillin, a cephalosporin, a fluroquinolone, a tetracycline, or a macrolide.

The formulation may be aerosolized into particles having an aerodynamic diameter in a range from 2.0 microns to 12.0 microns or an aerodynamic diameter in a range from 4.0 microns to 10.0 microns, wherein the particles of a single delivery dose, as combined, have a total volume in a range of from 0.05 mL to 5.0 mL or a total volume in a range of from 0.1 mL to 3.0 mL.

The formulation may be manufactured for the treatment of hypercalcemia.

The formulation may comprise ciprofloxacin.

A method of intrapulmonary drug delivery is disclosed. The method includes administering an aerosolized formulation to a patient's respiratory tract by inhalation. The aerosolized formulation is comprised of particles which have a diameter in a range of about 0.5 microns to about 15 microns and more preferably 1 microns to 6 microns. The particles are comprised of a formulation designed for aerosolized delivery. The formulation is comprised of a pharmaceutically active drug, a pharmaceutically acceptable carrier and an agent which affects the pH of the formulation. The agent is added in a molarity so as to deviate the pH of the formulation away from 7.0. The deviation away from 7.0 to 8.0 or 6.0 which would be plus one log unit or minus one unit, respectively. The movement away from neutrality could be any fraction of a log unit e.g. 1/10, ¼, ½, ⅔, etc. Making the formulation highly basic (e.g. pH 10 or higher) or highly acidic (e.g. pH 2 or lower) could damage lung tissue, especially if large volumes of solution were inhaled, or if the solution had a high buffering capacity. Thus, for larger inhalation volumes the range that may be useful is pH 4.5 to pH 6.5 on the acidic side and pH 7.5 to 9.5 on the basic side. However for smaller inhaled volumes, or formulations with low buffering capacity, the useful range may expand to pH 1.5 to pH 6.5 on the acidic side and pH 7.5 to 10.5 on the basic side. The pH in human blood is about pH 7.4 which is slightly basic.

Agents which can be used to effect a change in pH include salts, acids, bases and other excipients which drive the equilibrium concentration of the hydrogen ion concentration either up or down. The addition of acids such as HCl (hydrochloric acid), phosphoric acid, acetic acid, citric acid, lactic acid, ascorbic acid, sulfuric acid, succinic acid, benzoic acid, lipoic acid and malic acid will tend to increase the concentration of the hydrogen ion thus resulting in a decrease in the solution pH which is defined as the negative of the log of the hydrogen ion concentration. In contrast, bases such as NaOH (sodium hydroxide) will tend to decrease the hydrogen ion concentration and thus increase the pH. Amino acids can be used to reduce the pH if the amino acid is in the hydrochloride form (e.g., aspartic hydrochloride or glycine hydrochloride), or increase the pH if the amino acid is in a salt form (e.g., disodium aspartate or sodium glyconate) Buffering agents, such as salts and amino acids, can also be used so that the pH in solution remains relatively constant and is less sensitive to perturbations.

After administering the formulation the formulation is allowed to remain in contact with the patient's respiratory tract fluids for a period of time and under conditions such that the formulation moves closer to a neutral pH. Specifically, the pH of the formulation will change by ±1 log unit, ±2 log units, ±3 log units or more relative to the pH of the formulation prior to administration.

By initially formulating the drug in a formulation which is either acidic or basic a greater amount of drug can be dissolved in the formulation. Stated differently the concentration of the drug in a solvent carrier can be increased by changing the pH away from neutrality. However, when the formulation comes in contact with the patient's respiratory tract fluids, it is designed such that the formulation can, to a degree, be quickly neutralized without causing a significant change in the local pH in the lung. This is achieved by formulations that have very low buffering capacity, i.e., only a small amount of acid or base is required to neutralize them. As the pH moves closer to neutrality the solubility of the drug is decreased and the drug may crystallize or precipitate out of solution depending on the solubility of that drug at the neutral or more nearly neutral pH. This provides for drug crystals or precipitate which can dissolve over a Jong period of time and thereby provide for long term controlled release of the drug to the patient. By initially dissolving the drug in a formulation which has a pH different from neutrality a larger amount of drug can be included in the formulation. This is desirable in that less aerosol needs to be delivered to the patient in order to obtain the desired therapeutic level of dosing.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Gallium is a semi-metallic element in group 13 (IIIa) of the periodic table. Gallium is trivalent in aqueous solution (Ga³⁺). The free hydrated ion Ga³⁺hydrolyzes nearly completely at pH values close to neutral, readily forming highly insoluble amorphous Ga(OH)₃. In addition to precipitating as hydroxides and oxyhydroxides , Ga will also form highly insoluble phosphates at pH values close to neutral. L R Bernstein (1998) provides a brief review of the solution chemistry of gallium. At pH 7.4 and 25° C. the total aqueous solubility of gallium is only ˜1 μM with the minimum solubility at pH 5.2 (10^{-7 2} M). At low and high pH values, gallium has many orders of magnitude greater solubility. For example, at pH 2, the solubility is ˜10² M which is ˜10,000 times greater solubility than at pH 7.4. Additionally, at pH 10, the solubility is ˜10^{-3.3} M which is ˜500 times greater solubility than at pH 7.4. This difference in solubility can be exploited in an inhalation product by formulating gallium or its salts (e.g., gallium nitrate) at a very low or very high pH.

For example, using the AERx® technology, one AERx® strip might contain 50 μL of a gallium inhalation solution near its solubility limit at pH 2 (˜10² M). Previous clinical trials using the AERx® technology have demonstrated lung delivery of 50% or more of the loaded drug dose in the dosage form. Assuming that 50% of the gallium deposits uniformly throughout the lung and that the 25 μL of gallium solution from one dosage form rapidly equilibrates to ˜pH 7.4 in 20 mL of lung fluid, the resulting gallium concentration (˜12.5 μM) would exceed its equilibrium solubility at pH 7.4 (˜1 μM) by ˜12.5 fold. This suggests that 96% of the gallium is likely to precipitate out of solution with only 8% remaining soluble. Thus, one would expect that there would be a depot-like effect in the lung in terms of the release of gallium from the solid state over time. This would result in a delayed absorption profile of gallium into the bloodstream, with a reduced C_{MAX} and delayed T_{MAX}. This would also reduce or eliminate side effects resulting from high systemic concentrations.

The judicious use of other formulation salts or excipients to further increase the solubility of gallium at these low or high pH values would result in an incremental increase in the dose that could be delivered in one puff yet likely not perturb the inherently poor solubility at pH 7.4. There are many potential excipients that could be used including surfactants, complexation agents including cyclodextrins and liposomal formulations. Additionally, suspensions of encapsulated gallium could also be designed using microparticles or polymeric materials such as PLGA to encapsulate gallium. The net effect of using suspensions would be to form insoluble particulates prior to delivery to the lung, yet retain an aqueous or liquid formulation allowing ease of inhalation delivery using a solution inhaler such as AERx.

### Example 2

The second example is the inhalation delivery of an anti-infective or antibiotic to more effectively treat lung infections or lung disease. Antibiotics may be informally defined as the sub-group of anti-infectives that are derived from bacterial sources and are used to treat bacterial infections. Other classes of drugs, most notably the sulfonamides, may be effective antibacterials. Similarly, some antibiotics may have secondary uses, such as the use of demeclocycline (Declomycin, a tetracycline derivative) to treat the syndrome of inappropriate antidiuretic hormone (SIADH) secretion. Other antibiotics may be useful in treating protozoa! infections.

Although there are several classification schemes for antibiotics, based on bacterial spectrum (broad versus narrow) or route of administration (injectable versus oral versus topical), or type of activity (bactericidal vs. bacteriostatic), the most useful is based on chemical structure. Antibiotics within a structural class will generally show similar patterns of effectiveness, toxicity, and allergic potential.

PENICILLINS. The penicillins are the oldest class of antibiotics, and have a common chemical structure which they share with the cephalopsorins. The two groups are classed as the beta-lactam antibiotics, and are generally bacteriocidal—that is, they kill bacteria rather than inhibiting growth. The penicillins can be further subdivided. The natural pencillins are based on the original penicillin G structure; penicillinase-resistant penicillins, notably methicillin and oxacillin, are active even in the presence of the [bacteria] enzyme that inactivates most natural penicillins. Aminopenicillins such as ampicillin and amoxicillin have an extended spectrum of action compared with the natural penicillins; extended spectrum penicillins are effective against a wider range of bacteria. These generally include coverage for Pseudo mon as aeruginaosa and may provide the penicillin in combination with a penicillinase inhibitor.

CEPHALOSPORINS. Cephalosporins and the closely related cephamycins and carbapenems, like the pencillins, contain a beta-lactam chemical structure. Consequently, there are patterns of cross-resistance and cross-allergenicity among the drugs in these classes. The “cepha” drugs are among the most diverse classes of antibiotics, and are themselves subgrouped into 1st, 2nd and 3rd generations. Each generation has a broader spectrum of activity than the one before. In addition, cefoxitin, a cephamycin, is highly active against anaerobic bacteria, which offers utility in treatment of abdominal infections. The 3rd generation drugs, cefotaxime, ceftizoxime, ceftriaxone and others, cross the blood-brain barrier and may be used to treat meningitis and encephalitis. Cephalopsorins are the usually preferred agents for surgical prophylaxis.

FLUROQUINOLONES. The fluroquinolones are synthetic antibacterial agents , and not derived from bacteria. They are included here because they can be readily interchanged with traditional antibiotics. An earlier , related class of antibacterial agents, the quinolones, were not well absorbed , and could be used only to treat urinary tract infections. The fluroquinolones, which are based on the older group, are broad-spectrum bacteriocidal drugs that are chemically unrelated to the penicillins or the cephaloprosins. They are well distributed into bone tissue, and so well absorbed that in general they are as effective by the oral route as by intravenous infusion.

TETRACYCLINES. Tetracyclines got their name because they share a chemical structure that has four rings. They are derived from a species of Streptomyces bacteria. Broad-spectrum bacteriostatic agents, the tetracyclines may be effective against a wide variety of microorganisms, including rickettsia and amebic parasites.

MACROLIDES. The macrolide antibiotics are derived from Streptomyces bacteria, and got their name because they all have a macrocyclic lactone chemical structure. Erythromycin, the prototype of this class, has a spectrum and use similar to penicillin. Newer members of the group, azithromycin and clarithyromycin, are particularly useful for their high level of lung penetration. Clarithromycin has been widely used to treat Helicobacter pylori infections, the cause of stomach ulcers.

OTHERS. Other classes of antibiotics include the aminoglycosides, which are particularly useful for their effectiveness in treating Pseudomonas aeruginos a infections; the lincosamindes, clindamycin and lincomycin, which are highly active against anaerobic pathogens. There are other, individual drugs which may have utility in specific infections.

It is anticipated that many anti-infectives or antibiotics may be amenable to improved treatment of lung infections by this invention. One example is inhaled tobramycin; e.g., TOBI, which is prescribed for cystic fibrosis and is administered twice a day in a format of 5 mL containing 60 mg/ml tobramycin. This is not a particularly convenient administration regime for patients and a more sustained release profile would allow less frequent dosing and potentially better efficacy at a lower dose with reduced side effects. While tobramycin is very soluble in water, other antibiotics indicated for topical treatment, like ofloxacin for ocular indications, has a solubility of less than about 3 mg/mL at neutral pH with lowest solubility for the zwitterionic species at pH 7. A decrease in the pH by two log units to pH 5 results in an increase in solubility to >95 mg/mL. Thus, it would be possible to formulate very high concentrations of ofloxacin at a pH less than 5 and upon inhalation delivery to the lung, and equilibration with the neutral pH of the lung, the antibiotic may precipitate out of solution allowing for a sustained depot-like release within the lung.

Another example is ciprofloxacin. Inhaled ciprofloxacin is under development for treatment of lung infections by a number of companies; Aradigm's liposomal ciprofloxacin hydrochloride and Bayer/Nektar's dry powder formulation of ciprofloxacin and pegylated ciprofloxacin. It is well known that ciprofloxacin bas its lowest solubility at neutral pH (pH 7.4) and exists as a zwitterionic species . The solubility of ciprofloxacin hydrochloride at pH 7 is less than 0.1 mg/mL. At pH values substantially away from neutrality, the solubility increases exponentially to greater than 20 mg/mL at low and high pHs. This characteristic can be exploited to formulate a high concentration ciprofloxacin hydrochloride solution at either very low (pH<4) or very high pH (pH>9). Upon inhalation of the high concentration ciprofloxacin formulation and deposition in the lung milieu, the ciprofloxacin will rapidly be equilibrated to neutral pH. This may cause the ciprofloxacin hydrochloride, or other ciprofloxacin salts, to precipitate out of solution and or form crystals. These insoluble crystals or precipitates will slowly dissolve over time attenuating the release of ciprofloxacin in the lung, and thus reducing and prolonging the absorption into the bloodstream; i.e., lowering the C_{MAX} and increasing the T_{MAX}.

The total lung fluid is thought to be about 20 mL in adult humans. If the amount of ciprofloxacin delivered to the lung exceeds a few mg, then the ciprofloxacin concentration will exceed its solubility at neutral pH. Depending upon the specific aerosol delivery methodology and inhalation parameters, it is also possible that the aerosol droplets will not deposit uniformly throughout the lung. This concept may be exploited to advantage by allowing delivery of even lower amounts of the antibiotic while still resulting in the local concentration of ciprofloxacin exceeding The solubility limit in particular regions of the lung , either well-defined regionally; e.g., central or peripheral, or undefined depending upon where more droplets deposit. In either case, the result may be formation of ciprofloxacin structures that provide a depot like release of ciprofloxacin over time.

This example can be generally applied to other antibiotics which exhibit either improved solubility or stability at pH values away from neutrality.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, an statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method of treating a bacterial infection, comprising: aerosolizing a volume of 0.1 mL to 3.0 mL of formulation to create aerosolized particles having an aerodynamic diameter in a range of 4.0 microns to 10.0 microns;
administering the aerosolized particles to patient's respiratory tract by inhalation in a single delivery dose , wherein the formulation is comprised of ciprofloxacin, a pharmaceutically acceptable carrier and a pH affecting agent which increases solubility of the ciprofloxacin in the carrier and is present in a molarity so as to result in a pH of 3.3; and
allowing the formulation to remain in contact with the patient's respiratory tract fluids for a period of time and under conditions such that the formulation moves closer to a pH of 7.4 by 0.5 log unit or more relative to the pH of the formulation prior to administration;
wherein the ciprofloxacin becomes less soluble as compared to its solubility in the formulation prior to administration.

2. A method of treating a bacterial infection , comprising: aerosolizing a volume of 0.1 mL to 3.0 mL of formulation to create aerosolized particles having an aerodynamic diameter in a range of 4.0 microns to 10.0 microns;
administering the aerosolized particles to patient's respiratory tract by inhalation in a single delivery dose, wherein the formulation is comprised of liposomes, ciprofloxacin, a pharmaceutically acceptable carrier and a pH affecting agent which increases solubility of the ciprofloxacin in the carrier and is present in a molarity so as to result in a pH of 3.3; and
allowing the formulation to remain in contact with the patient's respiratory tract fluids for a period of time and under conditions such that the formulation moves closer to a pH of 7.4 by 0.5 log unit or more relative to the pH of the formulation prior to administration;
wherein the ciprofloxacin becomes less soluble as compared to its solubility in the formulation prior to administration.

3. A method of treating a bacterial infection, comprising:
aerosolizing a volume of 0.1 mL to 3.0 mL of formulation to create aerosolized particles having an aerodynamic diameter in a range of 4.0 microns to 10.0 microns;
administering the aerosolized particles to patient's respiratory tract by inhalation in a single delivery dose, wherein the formulation is comprised of ciprofloxacin at a concentration of 20 mg/mL or more, a pharmaceutically acceptable carrier and a pH affecting agent which increases solubility of the ciprofloxacin in the carrier and is present in a molarity so as to result in a pH of 3.3; and allowing the formulation to remain in contact with the patient's respiratory tract fluids for a period of time and under conditions such that the formulation moves closer to a pH of 7.4 by 0.5 log unit or more relative to the pH of the formulation prior to administration;
wherein the ciprofloxacin becomes less soluble as compared to its solubility in the formulation prior to administration.
